(19) United States
(12) Patent Application Publication
(10) Pub. No.: US 2007/0110750 A1
(43) Pub. Date: May 17, 2007

(54) Monoclonal antibodies specific for high molecular weight aggregation intermediates common to amyloids formed from proteins of differing sequence

(75) Inventors: Charles G. Glabe, Irvine, CA (US); Rakez Kayed, Irvine, CA (US)

Correspondence Address: Stout Uxa;Buyan & Mullins, 4 Venture Suite 300, Irvine, CA, 92618, US

(73) Assignee: The Regents of the University of California, Oakland, CA (US)

(21) Appl. No.: 10/572,001

(22) PCT Filed: Sep. 13, 2004
(86) PCT No.: PCT/US04/29946

§ 371 Date: Dec. 21, 2006

Related U.S. Application Data
(60) Provisional application No. 60/502,326, filed on Sep. 12, 2003.

(51) Int. Cl. A61K 39/395; G01N 33/567; C07K 16/40; G01N 33/53

(52) U.S. Cl. 424/146.1; 530/388.26; 424/178.1; 435/7.2; 977/906

(57) ABSTRACT

Methods for the production of monoclonal antibodies specific to conformational epitope(s) of a prefibrilar aggregate(s) which contribute to amyloid fibril formation in human or animal subjects who suffer from amyloid diseases (e.g. Alzheimer's Disease) and the hybridomas and monoclonal antibodies produced therefrom. Also, the use of such monoclonal antibodies in the immunization of human or animal subjects against Alzheimer's Disease or other amyloid diseases and/or for the diagnosis or detection of Alzheimer's Disease or other amyloid diseases. The monoclonal antibodies may be administered concomitantly or in combination with anti-inflammatory agents, such as gold or gold containing compounds, to decrease neural inflammation associated with amyloid diseases (e.g. Alzheimer's Disease).

### RELATED APPLICATION

This patent application claims priority to U.S. Provisional Patent Application No. 60/502,326 filed on Sep. 12, 2003, the entirety of which is expressly incorporated herein by reference.

### FIELD OF THE INVENTION

This invention relates generally to the fields of medicine, immunology and protein biochemistry and more particularly to a) methods for the production of monoclonal antibodies specific to conformatonal epitope(s) of a prefibrillar aggregate(s) which contribute to amyloid fibril formation in human or animal subjects, b) the hybridomas and monoclonal antibodies produced therefrom, c) the use of such monoclonal antibodies in the immunization of human or animal subjects against Alzhiemer's Disease or other amyloid diseases and d) the use of such monoclonal antibodies in the diagnosis or detection of Alzhiemer's Disease or other amyloid diseases in human or animal subjects.

### BACKGROUND OF THE INVENTION

Many biological functions come about, at least in part, due to the ability of proteins to adopt various sequence-dependent structures. However, certain protein sequences can sometimes form aberrant, misfolded, insoluble aggregates known as amyloid fibrils. These amyloid fibrils are thought to be involved in the pathogenesis of various amyloid diseases of genetic, infectious and/or spontaneous origin, including spongiform encephalopathies, Alzheimer's disease, Parkinson's disease, type II diabetes, Creutzfeldt-Jakob disease, Huntington's disease, possibly macular degeneration, various prion diseases and numerous others. In at least some of these amyloid diseases, amyloid fibrils lead to the development of amyloid plaques.

Amyloid peptides are the principal constituent of amyloid plaques. In the case of Alzheimer's disease, the peptides are termed A(3 or (3-amyloid peptide. A peptide is an internal fragment of 39 to 43 amino acids of amyloid precursor protein (APP). Several mutations within the APP protein have been correlated with the presence of AD. See, for example, Goate et al., Nature, (1991) 349, 704 (valine to isoleucine); Chartier Marian et al., Nature (1991)353,844 (valine to glycine); Murrell et al. Science (1991) 21,97 (valine to phenylalanine); Mullan et al., Nature Genet. (1992) 1,345 (a double mutation changing lysine 595-methionine596.to asparagine595-leucine596). Such mutations are thought to cause AD by producing an increased or altered processing of APP to Aβ. In particular, the processing of APP resulting in accumulation of the longer forms of Aβ, for example, Aβ1-42 and Aβ1-43 is thought to be important in the cause of AD. Mutations in other genes, such as the presenilin genes PS1 and PS2, are thought to indirectly affect processing of APP resulting in production of the long form of Aβ. See, for example, Hardy, TINS (1997) 20,11.

It is believed that cytotoxic amyloid-beta peptide aggregates disrupt the integrity of cell membranes and elaborate reactive oxygen intermediates, thereby giving rise to elevations in cytosolic calcium and eventual cell death. Cell surface receptors for amyloid-beta peptide may also activate signal transduction mechanisms.

European Patent Publication EP 526,511 (McMichael) and PCT International Patent Publication WO/9927944 (Schenk) have described the administration of Aβ to patients for the treatment or prevention of Alzheimer's. However, although active immunization of Aβ to transgenic mice produces apparent benefits, the extension of this approach to AD patients has resulted in undesirable inflammation of the central nervous system in some of the subjects. See Hardy, D. J. Selkoe (2002) Science 297, 353-356. Soluble Aβ includes Aβ monomers as well as aggregations of such monomers referred to as prefibrillar aggregates. These prefibrillar aggregates lead to the development of amyloid fibrils.

Soluble Aβ content of the human brain is better correlated with the severity of AD than is the accumulation of amyloid plaques. See, for example, Y. M. Kuo et al. (1996) J. Biol. Chem. 271, 4077-4081; C. A. McLean et al. (1999) Annals of Neurology 46, 860-6; L. F. Lue et al. (1999) American Journal of Pathology 155, 853-862. In addition, recent reports suggest that the toxicity of A and other amyloidogenic proteins lies not in the soluble monomers or insoluble fibrils that accumulate, but rather in the prefibrillarprefibrillar aggregates. See, for example, Hartley et al. (1999), Journal of Neuroscience 19, 8876-8884; Lambert et al., Proceedings of the National Academy of Sciences of the United States of America (1998) 95, 6448-53; and Bucciantini et al., Nature (2002) 416, 507-511; and Hartley et al. Nature (2002) 418, 291. Taken together, these results indicate that the prefibrillar aggregates may be more pathologically significant than other forms of the amyloid peptides and therefore may be a more desirable target in the prevention or curing of amyloid diseases such as AD.

PCT International Patent Application PCT/US2003/028829 (WO 2004/024090) entitled MONOCLONAL ANTIBODYS AND CORRESPONDING ANTIBODIES SPECIFIC FOR HIGH MOLECULAR WEIGHT AGGREGATION INTERMEDIATES COMMON TO AMYLOIDS FORMED FROM PROTEINS OFDIFFERING SEQUENCE (Kayed and Glabe) describes compositions of matter comprising one or more conformational epitopes found on amyloid peptide aggregates, antibodies to such epitopes and methods for making and using the compositions, epitopes and/or antibodies. The compositions described in PCT/US2003/028829 include synthetic or isolated compositions that contain or consist of certain conformational epitopes found on peptide aggregates (e.g., toxic peptide aggregates) present in human or veterinary patients who suffer from, or who are likely to develop, amyloid diseases (e.g., Alzheimers Disease). The invention described in PCT/US2003/028829 also includes methods for using such compositions in the detection, treatment and prevention of diseases in humans or animals and/or in the testing and identification of potential therapies (e.g., drug screening) using such antibodies. The entirety of PCT International Patent Application PCT/US2003/028829 is expressly incorporated herein by reference.

Monoclonal antibodies are homogeneous preparations of immunoglobulin proteins that specifically recognize and bind to regions, or epitones, of their corresponding antigens. In some cases, monoclonal antibodies can bind to and inhibit the activity of endogenous chemical entities that are toxic or deleterious. In view of this, there is a need for the development of new monoclonal antibodies that bind to and inhibit toxic forms of amyloid (e.g., cytotoxic amyloid-beta peptide aggregates or protofibrils) with high specificity, thereby providing for diagnosis and treatment of amyloid diseases.

### SUMMARY OF THE INVENTION

The present invention provides compositions comprising isolated monoclonal antibodies which bind to one or more conformational epitope(s) of prefibrillar aggregate(s) that contribute to amyloid fibril formation in the brains of humans or animals (e.g., toxic species of prefibriliar aggregate(s)). The monoclonal antibodies may be administered, in therapeutic amounts, to human or animal subjects to reduce the toxicity of the prefibrillar aggregate, thereby preventing or limiting the formation of amyloid deposits and the associated occurrence or progression of a disease or disorder in which amyloid deposits form within the brain or nervous tissue. Examples of such amyloid diseases include, but are not necessarily limited to, Alzheimer's Disease, early onset Alzheimer's Disease associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis. The monoclonal antibodies of the present invention are identified as follows: 354B85.1 (clone #56), 354B85.1 (clone #38), 354B85.1 (clone #45), 354B133, 354B256, and 354B273. These clones were prepared by immunizing mice with a conformationally-constrained antigen consisting of amyloid Aβ covalently coupled to colloidal gold via a thioester linkage.

In accordance with the invention, the prefibrillar aggregate may have a molecular weight in a range of about 1 kDa to about 100,000,000 kDa. Also, the prefibrillar aggregate may comprise any suitable number of monomers. For example, In some specific embodiments the prefibrillar aggregate may comprise five monomers and in other embodiments, the prefibrillar aggregate may comprise eight monomers.

Still further in accordance with the invention, the amyloid peptide monomers and/or amyloid fibrils may be substantially free of the conformational epitope to which the monoclonal antibody binds.

Still further in accordance with the invention, the monoclonal antibodies my be coupled to colloidal gold or may be administered concomitantly with gold or gold containing preparations to inhibit certain

Still further aspects and objects of the present invention may be understood from the detailed description and examples set forth herebelow.

### BRIEF DESCRIPTION OF THE DRAWINGS

FIG. 1 is a table comparing the effects of several monoclonal antibodies of the present invention.

FIG. 2 shows dot blot data obtained for several monoclonal antibodies of the present invention.

### DETAILED DESCRIPTION

### DEFINITIONS

As used in this patent application and/or in PCT International Application PCT/US2003/028829 (Publication No. WO 2004/024090 A2) which is incorporated by reference, the following terms shall have the following meanings:

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

The term “ [symbol] ” “ [symbol] peptide” refers to peptides which comprise low molecular weight soluble oligomers, prefibrillar aggregates, fibrils and amyloid deposits each associated with AD. Amyloid [symbol] peptides include, without limitation, [symbol] 39, [symbol] 40, [symbol] 41 [symbol] 42 and [symbol] 43 which are 39, 40, 41, 42 and 43 amino acid amino acids in length, respectively.

An “amyloid peptide” is a peptide that is present in amyloid forms including amyloid peptide intermediates, low molecular weight soluble oligomers, amyloid fibrils and amyloid plaques.

The term “antibody” is used to include intact antibodies and binding fragments thereof, including but not limited to, for example, full-length antibodies (e.g., an IgG antibody) or only an antigen binding portion (e.g.,a Fab, F(ab′)₂ or scFv fragment). Typically, fragments compete with the intact antibody from which they were derived for specific binding to an antigen. Optionally, antibodies or binding fragments thereof, can be chemically conjugated to, or expressed as, fusion proteins with other proteins. “Anti-oligomer antibody” or “Anti-oligomer” refer to an antibody that binds to amyloid peptide aggregate intermediates but does not bind to or does not specifically bind to amyloid peptide monomers, dimers, trimers or tetramers.

Compositions or methods “comprising” one or more recited elements may include other elements not specifically recited. For example, a composition that comprises an amyloid [symbol] peptide may encompass both an isolated amyloid A peptide as a component of a larger polypeptide sequence or as part of a composition which includes multiple elements.

The term “epitope” or “antigenic determinant” refers to a site on an antigen to which B and/or T cells respond or a site on a molecule against which an antibody will be produced and/or to which an antibody will bind. For example, an epitope can be recognized by an antibody defining the epitope.

A “linear epitope” is an epitope wherein an amino acid primary sequence comprises the epitope recognized. A linear epitope typically includes at least 3, and more usually, at least 5, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the antibody defining the epitope). Typically a conformational epitope comprises an increased number of amino acids relative to a linear epitope. With regard to recognition of conformational epitopes, the antibody recognizes a 3-dimensional structure of the peptide or protein. For example, when a protein molecule folds to form a three dimensional structure, certain amino acids and/or the polypeptide backbone forming the conformational epitope become juxtaposed enabling the antibody to recognize the epitope. Methods of determining conformation of epitopes include but are not limited to, for example, x-ray crystallography 2-dimensional nuclear magnetic resonance spectroscopy and site-directed spin labeling and electron paramagnetic resonance spectroscopy. See, for example, Epitope Mapping Protocols in Methods in Molecular Biology, Vol. 66, Glenn E. Morris, Ed. (1996), the disclosure of which is incorporated in its entirety herein by reference.

The term “immunological response” or “immune response” relates to the development of a beneficial humoral (antibody mediated) and/or a cellular (mediated by antigen-specific T cells or their secretion products) response directed against an amyloid peptide in a recipient patient. Such a response can be an active response induced by administration of monoclonal antibody or a passive response induced by administration of antibody or primed T-cells. A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity.

An “monoclonal antibodyic agent” or “monoclonal antibody” or “antigen” is capable of inducing an immunological response against itself upon administration to asubject, optionally in conjunction with an adjuvant.

“Isolated” means purified, substantially purified or partially purified. Isolated can also mean present in an environment other than a naturally occurring environment. For example, an antibody that is not present in the whole blood serum in which the antibody would ordinarily be found when naturally occurring is an isolated antibody.

“Low molecular weight aggregate”, “low molecular weight amyloid aggregate”, “low molecular weight oligomer” and “low molecular weight soluble oligomer” refer to amyloid peptides present in aggregates of less than four or five peptides. In one specific example, low molecular weight [symbol] refers to the low molecular weight soluble oligomers found associated with AD.

The term “patient” includes human and other animal subjects that receive therapeutic, preventative or diagnostic treatment or a human or animal having a disease or being predisposed to a disease.

“Prefibrillar aggregates”, “micellar aggregates”, “high molecular weight aggregation intermediates,” “high molecular weight amyloid peptide aggregates”, “high molecular weight soluble amyloid peptide aggregates” “amyloid peptide aggregates”, “soluble aggregate intermediates”, “amyloid oligomeric intermediates”, “oligomeric intermediates” and “oligomeric aggregates” or simply, “intermediates” refer to aggregations which include more than three individual peptide or protein monomers, for example, more than four peptide or protein monomers. The upper size of prefibrillar aggregates includes aggregations of oligomers which form spherical structures or micelles and stings of micelles which lead to fibril formation.

“Annular protofibrils” are a particular subset of prefibrillar aggregates in which 3 to 10 spherical oligomer subunits are arranged in an annular or circular fashion with a hollow center that appears as a pore in electron or atomic force micrographs.

The molecular weight of a prefibrillar aggregate may be in a range of about 10 kDa to about 100,000,000 KDa, for example, about 10 kDa to about 10,000,000 or 1,000,000 KDa. However, this size range is not intended to be limiting and prefibrillar aggregates are not defined by a molecular weight range.

“Protofibrils” are prefibrillar aggregates which include spherical structures comprising amyloid [symbol] peptides that appear to represent strings of the spherical structures forming curvilinear structures.

“Specific binding” between two entities means an affinity of at least 10⁶, 10⁷, 10⁸ 10⁹ M⁻¹, or 10¹⁰ M⁻¹. Affinities greater than 10⁸ M⁻¹ are preferred for specific binding.

The term “substantial identity” means that two peptide sequences, when optimally aligned, such as by the programs GAP or BESTFIT using default gap weights, share at least 65 percent sequence identity, for example, at least 80 percent or 90 percent sequence identity, or at least 95 percent sequence identity or more, for example, 99 percent sequence identity or higher.

Preferably, residue positions in an alignment which are not identical differ by conservative amino acid substitutions, i.e., substitution of an amino acid for another amino acid of the same class or group. Some amino acids may be grouped as follows: Group I (hydrophobic side chains): leu, met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe. Non-conservative substitutions may include exchanging a member of one of these classes for a member of another class.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm may then be used to calculate the percent sequence identity for the test sequence (s) relative to the reference sequence, based on the designated program parameters. Optimal alignment of sequences for comparison can be conducted, for example, by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix, see for example, Henikoff & Henikoff, Proc. Natl. Acad. Sci. USA 89,10915 (1989). Conservative substitutions involve substitutions between amino acids in the same class.

A “therapeutic agent” or “therapeutic” is a substance useful for the treatment or prevention of a disease in a patient. Therapeutic agents of the invention are typically substantially pure. This means that an agent is typically at least about 50% w/w (weight/weight) pure, as well as being substantially free from proteins and contaminants which interfere with the efficacy of the therapeutic. The agents may be at least about 80% w/w and, more preferably at least 90 % w/w or about 95% w/w in purity. However, using conventional protein purification techniques, homogeneous peptides of 99% w/w or more can be produced.

### EMBODIMENTS AND EXAMPLES

Amyloid diseases are characterized by the accumulation of amyloid plaques or precursors to amyloid plaques in patients or the predisposition to the accumulation of amyloid plaques or precursors to amyloid plaques in patients. One of the primary constituents of amyloid plaques are amyloid peptides. The general conformation of amyloid peptides may vary from disease to disease, but often the peptide has a characteristic-pleated sheet structure. Amyloid peptides include peptides and proteins of about 10 or about 20 amino acids to about 200 amino acids in length. Though this size range is not intended as a limitation and amyloid peptides or proteins having fewer or more amino acids are contemplated in the present invention.

Prefibrillar aggregates are intermediates in the production of insoluble fibrils that accumulate in amyloid plaques of humans or animals having a disease characterized by amyloid deposits, for example, Alzheimer's disease. Prefibrillar aggregates include aggregates which may be as small as four amyloid peptides, as small as five amyloid peptides, as small as six amyloid peptides, as small as seven amyloid peptides or as small as eight amyloid peptides. In one embodiment, prefibrillar aggregates are micellar aggregates or micelles or strings of micelles. Prefibrillar aggregates are effective to form a conformational epitope which is recognized by an antibody of the present invention.

The conformational epitopes found on prefibrillar aggregates are substantially not found in the native precursor proteins for amyloid peptides, for example, amyloid peptide monomers, dimers, trimers or tetramers nor in the mature amyloid fibers that are defined by their characteristic cross x-ray fiber diffraction pattern or in amyloid plaques. The prefibriliar aggregates that contain the specific polypeptide structure which results in conformational epitopes that are recognized by antibodies of the present invention have a size range of approximately a pentamer, a hexamer, a heptamer or an octamer to micellar forms or protofibrils which have a molecular weight in excess of 1,000,000 Daltons. Antibodies of the invention are effective to bind to these epitopes.

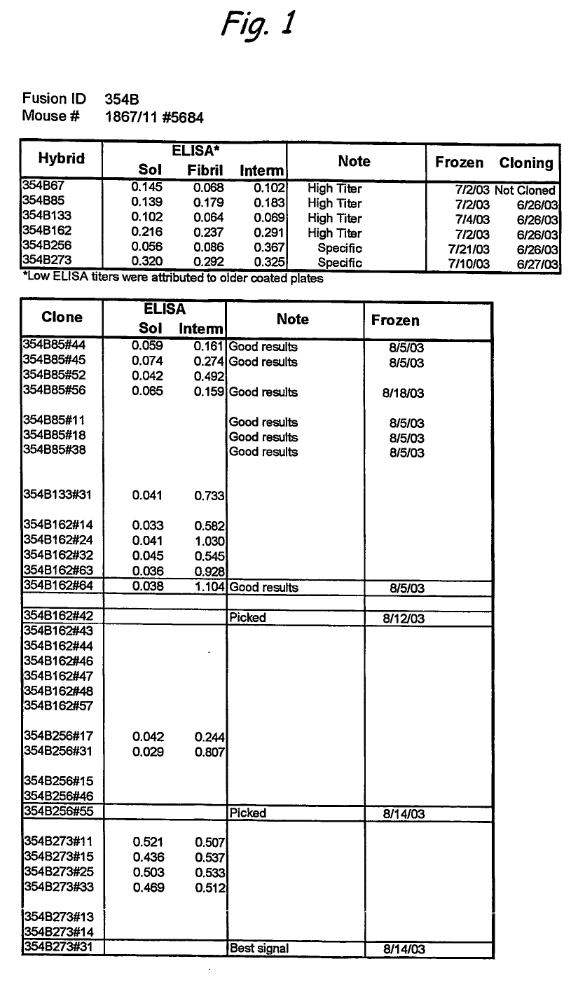

Monoclonal antibodies of the present invention are specific for a conformation-dependent epitope associated with amyloid oligomers or protofibrils. The monoclonal antibodies may be prepared by immunizing mice with a conformationally-constrained antigen consisting of amyloid Aβ covalently coupled to colloidal gold via a thioester linkage. FIG. 1 shows in diagrammatic form an example of how such monoclonal antibodies may be produced. Such monoclonal antibodies will provide for diagnostic and therapeutic uses. The antibody is also useful for determining the three dimensional structure of amyloid oligomers bound to the antibody by co-crystallization of the antibody Fab with the antigen and X-ray crystallography.

Supernatiants from hybridoma fusions were screned by ELISA by Strategic Biosolutions and the same supernatants were sent to UCI for further analysis by Dr. Rakez Kayed, Monica Siegenthaler and Maya Hatch by dot blot assay. For ELISA assay, 100 ng of soluble oligomeric or fibrillar Aβ42 was suspended in plating buffer and used to coat hyBond ELISA plates for 1 hr to overnight. After coating the wells were blocked with 300 ul 10 BSA in Tris-buffered saline, 0.01% Tween 40 (TBST) at 37 degrees C. for 1 hr. Tissue culture supernatant from the hybridomas was added to the wells at 1:200, 1:500, 1:1000, 1:2000 and 1:5000 and incubated at 37 degrees for 1 hr. The plates were washed 3× with phosphate buffered saline (PBS) and 100 ul of goat anti mouse-horseradish peroxidase conjugate 1:10,000 dilution was added to each well and incubated for 1 hr. the plates were washed 3 times with PBS and then assayed for HRP activity by adding 100 ul of color diction substrate, TMB. The plates were read at 450 nm. Clones that show high reactivity against oligomers and low reactivity against monomer and fibrils were selected.

Dot Blot Assay:

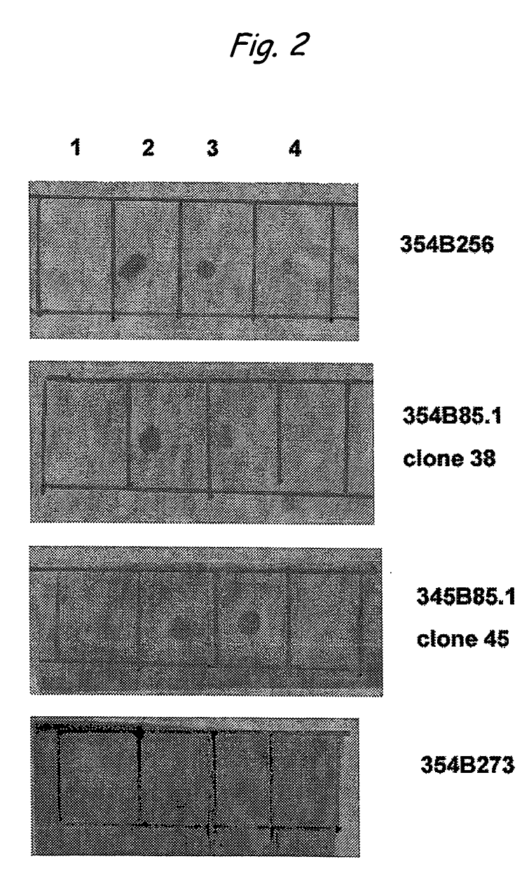

Monomer, oligomer and fibrillar samples of Aβ42 (100 ng) were applied to a nitrocellulose membrane, dried and blocked with 10% BSA in TBST. Tissue culture supernatant from the hybridomas was added to each strip at 1:200, 1:500, 1:1000, 1:2000 and 1:5000 and Incubated at 37 degrees for 1 hr. The strips were washed 3 times with PBS, and incubated at 37 degrees for 1 hr with goat anti mouse-horseradish peroxidase conjugate 1:10,000. The strips were washed 3 times with PBS and the antibody binding visualized by enhanced chemiluminescence (ECL). A typical dot blot is shown in FIG. 2 for clones 354B85.1 clone #38, and 354B85.1 clone #45, 354B256, and 354B273. Lane 1 is Aβ42 monomer. Lane 2 is Aβ42 oligomers. Lane 3 is Aβ42 fibrils. Lane 4 is human lysozyme oligomers.

FIG. 1 contains a summary of results pertaining to the screening of antibodies that are specific for a conformational epitope that is common to amyloid oligomeric intermediates. It summarizes the results of fusion 31 B from mouse 1867/11 #5684 that was vaccinated with a micelle molecular mimic consisting of a conformationally constrained Aβ40 thioester coupled to colloidal gold. The mouse was boosted with soluble Aβ40 oligomers 3 days before the spleen was removed and used for hybridoma production in order to increase the number of circulating B cells to useable levels. The first column lists the hybridoma clone label. The second column lists the results of ELISA assay using ELISA plates containing rows of soluble low MW Aβ (sol), oligomeric intermediates (interm) and amyloid fibrils (fibril). The numbers are optical absorbance values in absorbance units and represent the extent to which the different clones recognize the different conformations of the A(3 adsorbed to the plate. A low or background number in the sol and fibril column indicates a lack of binding or recognition, while a high value in the interm column indicates a high degree of recognition or binding. Clones with a low number for sol and fibrils with a high number for interm indicate a high degree of specificity for the soluble oligomer conformation dependent epitope. Examples of clones exhibiting a high degree of specificity for soluble oligomers and not low MW soluble Aβ or fibrils include, but are not limited to clones 354B85.1 (clone #56), 354B85.1 (clone #38), 354B85.1 (done #45), 354B256 and 354B273

Each of the following amyloid peptides have been shown to form amyloid peptide aggregates which produce a conformational epitope recognized by the antibodies of the present invention, for example, antibodies produced against [symbol] peptide oligomeric intermediates. Some of these peptides are present in amyloid deposits of humans or animals having a disease characterized by the amyloid deposits. The present invention is not limited to the listed peptide or protein sequences or the specific diseases associated with some of the sequences. The present invention contemplates antibodies as described herein binding to other amyloid peptide aggregates or all other amyloid peptide aggregates. In particular, the present invention contemplates and includes the application of methods and compositions of the present invention to other peptide or protein sequences which form amyloid precursor aggregates associated with other diseases.

A40 (SEQ ID NO 1)
DAEFRHDSGYEVHHQKLVFF AEDVGSNKGA IIGLMVGGVV
A42 (SEQ ID NO 2)
DAEFRHDSGY EVHHQKLVFF AEDVGSNKGA IIGLMVGGVV IA
Human IAPP (SEQ ID NO 3)
KCNTATCATQ RLANFLVHSS NNFGAILSST NVGSNTY
Human Prion 106-126 (SEQ ID NO 4)
KTNMKHMAGA AAAGAVVGGL G

Stefani and coworkers (Bucciantini et al (2002) Nature 416, 507-511) have recently reported that amyloid peptide aggregates formed from non-disease-related proteins are inherently cytoxic, suggesting that they may have a structure in common with disease related amyloid peptides. Non-disease related amyloid peptide aggregates comprising the following non-disease related amyloid peptides are also shown to bind to the antibodies of the present invention.

Poly glutamine synthetic peptide KK(Q40)KK
(SEQ ID NO 5)
KKQQQQQQQQ QQQQQQQQQQ QQQQQQQQQQ QQQQQQQQQQ
QQKK
Human Lysozyme (SEQ ID NO 6)
MKALIVLGLV LLSVTVQGKV FERCELARTL KRLGMDGYRG
SLANWMCLA KWESGYNTRA TNYNAGDRST DYGIFQINSR
YWCNDGKTPG AVNACHLSCS ALLQDNIADA VACAKRVVRD
PQGIRAWVAW RNRCQNRDVR QYVQGCGV
Human Insulin (SEQ ID NO 7)
MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN
Human Transthyretin (SEQ ID NO 8)
MASHRLLLLC LAGLVFVSEA GPTGTGESKC PLMVKVLDAV
RGSPAINVAV HVFRKAADDT WEPFASGKTS ESGELHGLTT
EEEFVEGIYK VEIDTKSYWK ALGISPFHEH AEVVFTANDS
GPRRYTIAAL LSPYSYSTTA VVTNPKE
Human Alpha Synuclein (SEQ ID NO 9)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

In addition, oligomeric intermediates formed from variants and fragments of wild type [symbol] 42, [symbol] 40 including, without limitation [symbol] 42 (A21G) Flemish mutation), [symbol] 42 (E22Q) Dutch mutation, [symbol] 42 (E22G) Arctic mutation, [symbol] 42 (D23N) Iowa mutation, [symbol] 40 (A21G) Flemish mutation), [symbol] 40 (E22Q) Dutch mutation, [symbol] 40 (E22G) Arctic mutation, [symbol] 40 (D23N) Iowa mutation, [symbol] 40 (E22Q &D23N) Dutch & !Iowa mutations, [symbol] 3-42 (pGlu 3), [symbol] 3-40 (pGlu 3), [symbol] 8-42, [symbol] 17-42, [symbol] 1-16, [symbol] 3-11, [symbol] 25-35, [symbol] 4-16 (3 analogues, Cys¹⁶ [symbol] 4-16, 16, and Ala¹⁰ [symbol] 4-16 ), His6 [symbol] 40C40 (6 histidines appended to the amino terminus of AβC40) are recognized by the antibodies of the present invention. Other oligomeric intermediates recognized by antibodies of the invention include, without limitation, oligomeric intermediates formed from IAPP(C2AandC7A) where alanine is substituted for the naturally occurring cysteine in IAPP, Polyglutamine KKQ40KK or poly glutamine where the number of Q residues is greater than 32, Calcitonin, TTR and its mutants TTR Pro⁵⁵, TTR Phe⁷⁸, vitronictin, poly Lysine, poly arginine, serum amyloid A, cystantin C, IgG kappa light chain, oligomeric intermediates produced from other amyloid peptides disclosed herein and amyloid intermediates associated with amyloid diseases disclosed herein.

The present invention provides for amyloid disease therapeutics which induce a specific immune response against amyloid oligomeric intermediates. Therapeutics of the invention include antibodies that specifically bind to oligomeric intermediates. Such antibodies can be monoclonal as described in this application or polyclonal as described in PCT International Application No. PCT/US2003/028829, which is incorporated herein by reference. In one useful embodiment, the antibodies bind to a conformational epitope. The production of non-human monoclonal antibodies of the present invention (e.g., murine or rat) can be accomplished by, for example, immunizing the animal with an oligomeric intermediate mimic of the invention. Also contemplated is immunizing the animal with a purified amyloid intermediate.

Humanized forms of mouse antibodies of the invention can be generated by linking the CDR regions of non-human antibodies to human constant regions by recombinant DNA techniques. See Queen et al., Proc. Natl. Acad. Sci. USA 86,10029-10033 (1989) and WO 90/07861 (incorporated by reference for all purposes).

Human antibodies may be obtained using phage-display methods. See, for example, Dower et al., WO 91/17271 and McCafferty et al., WO 92/01047. In these methods, libraries of phage are produced in which members display different antibodies on their outer surfaces. Phage displaying antibodies with a desired specificity are selected by affinity enrichment. Human antibodies against oligomeric intermediates may also be produced from non-human transgenic mammals having transgenes encoding at least a segment of the human immunoglobulin locus and an inactivated endogenous immunoglobulin locus. See, for example, Lonberg et al., W093/12227 (1993); Kucherlapati, WO 91/10741 (1991) (each of which is incorporated by reference in its entirety for all purposes). Human antibodies can be selected by competitive binding experiments, or otherwise, to have the same epitope specificity as a particular mouse antibody. Such antibodies are particularly likely to share the useful functional properties of the mouse antibodies.

Human or humanized antibodies can be designed to have IgG, IgD, IgA and IgE constant region, and any isotype, including IgGI, IgG2, IgG3 and IgG4. Antibodies can be expressed as tetramers containing two light and two heavy chains, as separate heavy chains, light chains, as Fab, Fab′ F(ab′)₂ and Fv, or as single chain antibodies in which heavy and light chain variable domains are linked through a spacer.

In certain instances it may be desirable to combine one or more monoclonal anibodies of the present invention with a suitable carrier. Suitable carriers include serum albumins, keyhole limpet hemocyanin, immunoglobulin molecules, thyroglobulin, ovalbumin, tetanus toxoid, or a toxoid from other pathogenic bacteria, such as diphtheria, E. coli, cholera, or H. pylori, or an attenuated toxin derivative. Other carriers which may act as adjuvants for stimulating or enhancing an immune response include cytokines such as IL-1, IL-1 and peptides, IL-2, INF, IL-10, GM-CSF, and chemokines, such as MIP1 and and RANTES.

Human or animal subjects or patients amenable to treatment with monoclonal antibodies of the present invention include individuals at risk of amyloid disease but not showing symptoms, as well as those who already show symptoms or other evidence of amyloid disease. In the case of certain amyloid diseases including AD, virtually anyone is at risk of suffering from the disease.

Therefore, monoclonal antibodies of the present invention could be administered prophylactically, for example, as a vaccine, to the general population without any assessment of the risk of the subject patient. The present methods are especially useful for individuals who do have a known genetic risk of an amyloid disease, for example, AD. Such individuals may include those having relatives who have experienced an amyloid disease, and those whose risk is determined by analysis of genetic or biochemical markers or who exhibit symptoms or prodromes indicative of the potential for development of, or the actual presence of, such diseases. For example, genetic markers of risk toward AD include mutations in the APP gene, particularly mutations at position 717 and positions 670 and 671 referred to as the Hardy and Swedish mutations respectively (see Hardy, TINS, supra). Other markers of risk for AD are mutations in the presenilin genes, PSI and PS2, and ApoE4, family history of AD, hypercholesterolemia or atherosclerosis.

Symptoms of amyloid disease are apparent to a physician of ordinary skill. For example, Individuals presently suffering from Alzheimer's disease can be recognized from characteristic dementia, as well as the presence of risk factors described above. In addition, a number of diagnostic tests are available for identifying individuals who have amyloid diseases. For example, in the case of AD these include measurement of CSF tau and [symbol] 42 levels. Elevated tau and decreased [symbol] 42 levels signify the presence of AD.

In asymptomatic patients, treatment can begin at any age, for example, at the age of 10, 20, 30, 40, 50, 60 or 70. Treatment may entail one or more doses, for example, multiple dosages over a period of time. Treatment can be monitored by assaying antibody, or activated T-cell or B-cell responses to the therapeutic (for example, oligomeric intermediate mimic) or assaying the levels of prefibrillar aggregate present, each over time. In one embodiment, treatment by administering a single therapeutic of the invention, such as a preparation containing a single monoclonal antibody of the invention, may serve as a treatment for or preventive measure against more than one amyloid disease, for example all amyloid diseases.

In prophylactic applications, compositions of the invention or medians are administered to a patient susceptible to, or otherwise at risk of, a particular disease in an amount sufficient to eliminate or reduce the risk or delay the outset of the disease. In therapeutic applications, compositions or medians are administered to a patient suspected of, or already suffering from such a disease in an amount sufficient to cure, or at least partially arrest, the symptoms of the disease and its complications. An amount adequate to accomplish this is defined as a therapeutically-or pharmaceutically-effective dose. In both prophylactic and therapeutic regimes, therapeutics are usually administered in several dosages until a sufficient immune response has been achieved. Typically, the immune response is monitored and repeated dosages are given if the immune response starts to fade.

Effective doses of the compositions of the present invention, for the treatment of the above described conditions vary depending upon many different factors, including means of administration, target site, physiological state of the patient, whether the patient is human or animal, other medications administered, and whether treatment is prophylactic or therapeutic. Usually, the patient is a human, but in some diseases, such as mad cow disease, the patient can be a nonhuman mammal, such as a bovine or in the case of Alzheimer's disease, the patient may be a dog. Treatment dosages need to be titrated to optimize safety and efficacy. For passive immunization with an antibody, the dosage ranges from about 0.0001 mg/kg of body weight to about 100 mg/kg of body weight, and more usually about 0.01 mg/kg of body weight to about 5 mg/kg of body weight of the host. The amount of monoclonal antibody to be administered may depend on whether any adjuvant is also administered, with higher dosages being required in the absence of adjuvant. For example, 0.1 to 100 cc of a solution containing approximately 1% by weight of the desired monoclonal antibody(ies) my be injected subcutaneously, thereby delivering a dose of 1 mg to 1 g of the monoclonal antibody(ies) per injection. The timing of injections can vary significantly from once a day, to once a year, to once a decade. One typical regimen consists of an immunization followed by booster injections at 6 weekly intervals. Another regimen consists of an immunization followed by booster injections 1,2 and 12 months later. Another regimen entails an injection every two months for life. Alternatively, booster injections can be on an irregular basis as indicated by monitoring of immune response.

Therapeutics for inducing an immune response can be administered by any suitable route of administration, for example, parenteral, topical, intravenous, oral, subcutaneous, intraperitoneal, intranasal or intramuscular. The most typical route of administration is subcutaneous although others can be equally effective. The next most common is intramuscular injection. This type of injection is most typically performed in the arm or leg muscles. Intravenous injections as well as intraperitoneal injections, intraarterial, intracranial, or intradermal injections may also be effective in generating an immune response. In some methods, therapeutics are injected directly into a particular tissue where deposits have accumulated or may accumulate.

Monoclonal antibodies of the invention can optionally be administered in combination with other agents that are at least partly effective in treatment of amyloidogenic disease. In the case of Alzheimer's and Down's syndrome, in which amyloid deposits occur in the brain, therapeutics of the invention can also be administered in conjunction with other agents that increase passage of the compositions of the invention across the blood-brain barrier. For example, as described in detail herebelow, anti-inflammatory dosages of colloidal gold or gold salts may be administered concomitantly (e.g., before, concurrently with or after) the monoclonal antibody to deter the brain inflammation associated with AD and other amyloid diseases.

Monoclonal antibodies of the invention may sometimes be administered in combination with an adjuvant. A variety of adjuvants can be used in combination with an monoclonal antibody of the invention to elicit an immune response. Preferred adjuvants augment the intrinsic response to an monoclonal antibody without causing conformational changes in the monoclonal antibody that affect the qualitative form of the response. Preferred adjuvants include alum, 3 de-O-acylated monophosphoryl lipid A (MPL) (see GB 2220211). QS21 is a triterpene glycoside or saponin isolated from the bark of the Quillaja Saponaria Molina tree found in South America (see Kensil et al., in Vaccine Design: The subunit and Ajuvant Approach (eds. Powell & Newman, Plenum Press, NY, 1995); and U.S. Pat. No. 5,057,540). Other adjuvants are oil in water emulsions, such as squalene or peanut oil, optionally in combination with immune stimulants, such as monophosphoryl lipid A. See, for example, Stoute et al., N. Engl. J. Med. (1997) 336,86-91. Another useful adjuvant is CpG described in Bioworld Today, Nov. 15, 1998. Alternatively, a monoclonal antibody can be coupled to an adjuvant. However, such coupling should not substantially change monoclonal antibody so as to affect the nature of the immune response thereto. Adjuvants can be administered as a component of a therapeutic composition with an active agent or can be administered separately, before, concurrently with, or after administration of the therapeutic.

A preferred class of adjuvants is aluminum salts (alum), such as aluminum hydroxide, aluminum phosphate, aluminum sulfate. Such adjuvants can be used with or without other specific immunostimulating agents such as MPL or 3-DMP, QS21, polymeric or monomeric amino acids such as polyglutamic acid or polylysine.

Another class of adjuvants is oil-in-water emulsion formulations. Such adjuvants can be used with or without other specific immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-nPP) theramide™, or other bacterial cell wall components. Oil-in-water emulsions include (a) MF59 (WO 90/14837), containing 5% Squalene, 0.5% Tween 80 and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalane, 0.4% Tween 80,5% pluroinic-blocked polymer L121, and thr-MDP, either microfluidized into a submicron emulsion or vortexed to generate a larger particle size emulsion, and (c) Ribi™ adjuvant system (RAS), (Ribi Immunochem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™)

Another class of preferred adjuvants is saponin adjuvants, such as Stimulons (QS21, Aquila, Worcester, Mass.) or particles generated therefrom such as ISCOMs (immunostimulating complexes) and ISCOMATRIX. Other adjuvants include Complete Freund's Adjuvant (CFA) and Incomplete Freund's Adjuvant (IFA). Other adjuvants include cytokines, such as interleukins, for example, IL-1, IL-2, and IL-12, macrophage colony stimulating factor (M-CSF), tumor necrosis factor (TNF) and/or chemokines such as CXCL10 and CCL5.

An adjuvant can be administered with an monoclonal antibody as a single composition, or can be administered before, concurrent with or after administration of the monoclonal antibody. Monoclonal antibody and adjuvant can be packaged and supplied in the same vial or can be packaged in separate vials and mixed before use. Monoclonal antibody and adjuvant are typically packaged with a label indicating the intended therapeutic application. If monoclonal antibody and adjuvant are packaged separately, the packaging typically includes instructions for mixing before use. The choice of an adjuvant and/or carrier depends on the stability of the vaccine containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Optionally, two or more different adjuvants can be used simultaneously. Preferred combinations include alum with MPL, alum with QS21, MPL with QS21, and alum, QS21 and MPL together. Also, Incomplete Freund's adjuvant can be used (Chang et al., Advanced Drug Delivery Reviews 32,173-186 (1998)), optionally in combination with any of alum, QS21, and MPL and all combinations thereof.

Compositions of the invention are often administered as pharmaceutical compositions comprising a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonmonoclonal antibodyic stabilizers and the like. However, some reagents suitable for administration to animals, such as complete Freund's adjuvant are not typically included in compositions for human use.

Pharmaceutical compositions can also include large, slowly metabolized macromolecules such as proteins, polysaccharides, polylactic acids, polyglycolic acids and copolymers (such as latex functionalized sepharose, agarose, cellulose, and the like), polymeric amino acids, amino acid copolymers, and lipid aggregates (such as oil droplets or liposomes). Additionally, these carriers can function as immunostimulating agents (i. e., adjuvants).

For parenteral administration, compositions of the invention can be administered as injectable dosages of a solution or suspension of the substance in a physiologically acceptable diluent with a pharmaceutical carrier which can be a sterile liquid such as water oils, saline, glycerol, or ethanol.

Auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Compositions may be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. See Langer, Science (1990) 249, 1527and Hanes, Advanced Drug Delivery Reviews (1997) 28,97-119. The compositions of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to about 10%, for example, about 1% to about 2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and may contain about 10% about 95% of active ingredient, for example, about 25% to about 70%.

Topical application can result in transdermal or intradermal delivery. Topical administration can be facilitated by co-administration of the composition with cholera toxin or detoxified derivatives or subunits thereof or other similar bacterial toxins. See Glenn et al., Nature (1998) 391,851. Co-administration can be achieved by using the components as a mixture or as linked molecules obtained by chemical crosslinking or expression as a fusion protein.

Alternatively, transdermal delivery can be achieved using a skin path or using transferosomes. See for example, Paul et al., Eur. J. Immunol. (1995) 25,3521-24; Cevc et al., Biochem. Biophys. Acta (1998) 1368,201-15.

Concomitant Administration of Gold or Other Antiinflammatory

The anti-inflammatory effects of gold are well established. For example, injectable colloidal gold preparations (Myochrysine™ or Solganal™) are commercially available for the treatment of rheumatoid arthritis. A gold preparation for oral administration (Auranofin™) is also available. Inflammation of in the brain is thought to be a cause or contributing factor Alzheimer's Disease, primarily because amyloid-beta (protein) which is found in the brains of Alzheimer's patients is known to be an inflammatory protein. In view of this, others have proposed the use of non-steroidal anti-inflammatory drugs such as rofecoxib (Vioxx) and naproxen (Aleve) to slow the progression of Alzheimer's Disease.

Applicants have determined, on the basis of histopathological observations, that the subcutaneous administration of colloidal gold can reduce microglial activation in the brains of mice modeling for amyloid disease. The present invention includes the administration of colloidal gold, gold salts or other antiinflammatory agents to the subject in an amount that is therapeutically effective to decrease neural inflammation. In some cases, the gold or anti-inflammatory agent may be combined with the monoclonal antibody. In other cases, the gold or anti-inflammatory agent may be administered separately from the monoclonal antibody. Any syitable dose, dosing schedule or route of administration may be used. For example, commercially available gold preparations for treatment of rheumatoid arthritis may be administered by the same routes of administration (subcutaneous injection of Myochrysine™ or solganal™ or oral administration of Auranofin™ and dosages/dosing schedules recommended for treatment of rheumatoid arthritis.

Although the foregoing invention has been described in detail for purposes of clarity of understanding, it will be obvious that certain modifications may be practised within the scope of the appended claims. All publications and patent documents cited herein are hereby incorporated by reference in their entirety for all purposes to the same extent as if each were so individually denoted.

What is claimed is:

1. A composition comprising an isolated monoclonal antibody which binds to a conformational epitope of a prefibrillar aggregate which forms in a human or animal contributing to amyloid fibril formation, said monoclonal antibody being specific for a conformation-dependent epitope that is preferentially displayed by oligomeric conformations of Aβ and other amyloids.

2. A composition according to claim 1 wherein the monoclonal antibody is effective to reduce the toxicity of the prefibrillar aggregate.

3. A composition according to claim 1 wherein the prefibrillar aggregate has a molecular weight in a range of about 1 kDa to about 100,000,000 kDa.

4. A composition according to claim 1 wherein the prefibrillar aggregate comprises five monomers.

5. A composition according to claim 1 wherein the prefibrillar aggregate comprises eight monomers.

6. A composition according to claim 1 wherein amyloid peptide monomers are substantially free of the conformational epitope.

7. A composition according to claim 1 wherein amyloid fibrils are substantially free of the epitope.

8. A composition according to claim 1 wherein the prefibrillar aggregate comprises a toxic species.

9. A composition according to claim 1 wherein the prefibrillar aggregate is present in a human or animal having a disease characterized by amyloid deposits.

10. A composition according to claim 9 wherein the disease is selected from the group consisting of Alzheimer's Disease, early onset Alzheimer's Disease associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

11. A composition according to claim 9 wherein the disease is Alzheimer's.

12. A composition according to claim 1 wherein the composition is a pharmaceutical composition.

13. A preparation comprising at least one monoclonal antibody according to claim 1 in combination with at least one anti-inflammatory agent.

14. A preparation according to claim 13 wherein the anti-inflammatory agent comprises gold.

15. A composition comprising a monoclonal antibody which binds to an epitope of a prefibrillar aggregate which forms in a human or animal contributing to an amyloid fibril formation wherein the amyloid fibril is substantially free of the epitope.

16. A composition according to claim in 15 wherein the prefibrillar aggregate comprises a toxic species.

17. A composition according to claim 15 wherein amyloid peptide monomers are substantially free of the epitope.

18. A composition according to claim 15 wherein the monoclonal antibody is effective to reduce the toxicity of the prefibrillar aggregate.

19. A composition according to claim 15 wherein the prefibrillar aggregate has a molecular weight in a range of about 1 kDa to about 100,000,000 kDa.

20. A composition according to claim 15 wherein the prefibrillar aggregate comprises five monomers.

21. A composition according to claim 15 wherein the prefibrillar aggregate comprises eight monomers.

22. A composition according to claim 15 wherein the prefibrillar aggregate is present in a human or animal having a disease characterized by amyloid deposits.

23. A composition according to claim 22 wherein the disease is selected from the group consisting of Alzheimer's, early onset Alzheimer's associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

24. A composition according to claim 22 wherein the disease is Alzheimer's Disease.

25. A preparation comprising at least one monoclonal antibody according to claim 15 in combination with at least one anti-inflammatory agent.

26. A preparation according to claim 25 wherein the anti-inflammatory agent comprises gold.

27. A composition according to claim 15 wherein the composition is a pharmaceutical composition.

28. A method for treating a disease or condition characterized by amyloid deposits in a human or animal subject, said method comprising the step of:
A. causing a monoclonal antibody to bind to a conformational epitope of a prefibrillar aggregate which forms in a human or animal contributing to fibril formation.

29. A method according to claim 28 wherein step A comprises administering to the subject a therapeutically effective or preventative amount of a monoclonal antibody that has been prepared by immunizing mice with a conformationally-constrained antigen consisting of amyloid Ap covalently coupled to colloidal gold via a thioester linkage.

30. A method according to claim 28 wherein the prefibrillar aggregate comprises a toxic species of prefibrillar aggregate.

31. A method according to claim 30 wherein the monoclonal antibody is effective to reduce toxicity of the prefibrillar aggregate.

32. A method according to claim 28 wherein the prefibrillar aggregate has a molecular weight in a range of about 1 kDa to about 100,000,000 kDa.

33. A method according to claim 28 wherein the prefibrillar aggregate comprises five monomers.

34. A method according to claim 28 wherein the prefibrillar aggregate comprises eight monomers.

35. A method according to claim 28 wherein amyloid peptide monomers are substantially free of the epitope.

36. A method according to claim 28 wherein amyloid fibrils are substantially free of the epitope.

37. A method according to claim 28 wherein the prefibrillar aggregate is present in a human or animal having a disease characterized by amyloid deposits.

38. A method according to claim 28 wherein the disease or condition is selected from the group consisting of Alzheimer's Disease, early onset Alzheimer's Disease associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

39. A method according to claim 28 wherein the disease is Alzheimer's.

40. A method according to claim 28 wherein the composition is administered by a method selected from the group consisting of intraspinal, intrathecal, oral, transdermal, pulmonary, intravenous, subcutaneous, intranasal, intraarterial, intracranial, intradermal, intraperitoneal, intramuscular, rectal and buccal administration.

41. A method according to claim 28 further comprising the step of:
B. administering to the subject an antiinflamatory agent in an amount that is effective to deter brain inflammation.

42. A method according to claim 41 wherein Step B comprises administering gold or a gold-containing compound to the subject in an amount that is therapeutically effective to decrease neural inflammation.

43. A method according to claim 42 wherein a colloidal gold preparation is administered in Step B.

44. A method according to claim 41 wherein the anti-inflammatory agent is combined with the monoclonal antibody.

45. A method according to claim 41 wherein the anti-inflammatory agent is separate from the monoclonal antibody.

46. A method for treating a disease or condition characterized by amyloid deposits neural tissue in a human or animal subject, said method comprising the step of:
A. causing a monoclonal antibody to bind to an epitope of a prefibrillar aggregate which forms in a human or animal contributing to an amyloid fibril formation wherein the amyloid fibril is substantially free of the epitope.

47. A method according to claim 46 wherein step A comprises administering to the subject a therapeutically effective or preventative amount of a monoclonal antibody such that the monoclonal antibody will bind in accordance with Step A.

48. A method according to claim 46 wherein the monoclonal antibody binds to a conformational epitope of a prefibrillar aggregate that contributes to amyloid fibril formation in the human or animal subject, said monodonal antibody being specific for a conformation-dependent epitope that is preferentially displayed by oligomeric conformations of Aβ and other amyloids.

49. A method according to claim 46 wherein the prefibrillar aggregate has a molecular weight in a range of about 1 kDa to about 100,000,000 kDa.

50. A method according to claim 46 wherein the prefibrillar aggregate comprises five monomers.

51. A method according to claim 46 wherein the prefibrillar aggregate comprises eight monomers.

52. A method according to claim 46 wherein the prefibrillar aggregate comprises a toxic species.

53. A method according to claim 46 wherein the monoclonal antibody is effective to reduce toxicity of the prefibrillar aggregate.

54. A method according to claim 46 wherein amyloid fibrils are substantially free of the epitope.

55. A method according to claim 46 wherein the prefibrillar aggregate comprises a toxic species.

56. A method according to claim 46 wherein the prefibrillar aggregate is present in a human or animal having a disease characterized by amyloid deposits.

57. A method according to claim 46 wherein the disease or condition is selected from the group consisting of Alzheimer's, early onset Alzheimer's associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

58. A method according to claim 46 wherein the disease or condition is Alzheimer's Disease.

59. A method according to claim 46 wherein the composition is administered by a method selected from the group consisting of intraspinal, intrathecal, oral, transdermal, pulmonary, intravenous, subcutaneous, intranasal, intraarterial, intracranial, intradermal, intraperitoneal, intramuscular, rectal and buccal administration.

60. A method according to claim 46 further comprising the step of:
B. administering to the subject an antiinflamatory agent in an amount that is effective to deter brain inflammation.

61. A method according to claim 60 wherein Step B comprises administering gold or a gold-containing compound to the subject in an amount that is therapeutically effective to decrease neural inflammation.

62. A method according to claim 61 wherein a colloidal gold preparation is administered in Step B.

63. A method according to claim 62 wherein the anti-inflammatory agent is combined with the monoclonal antibody.

64. A method according to claim 63 wherein the anti-inflammatory agent is separate from the monoclonal antibody.

65. A method for making a monoclonal antibody, said method comprising the step of:
A. obtaining a conformational epitope of a prefibrillar aggregate which forms in a human or animal contributing to amyloid fibril formation.

66. The method according to claim 65 wherein step A comprises recovering the monoclonal antibody from a human or animal.

67. A method for making a monoclonal antibody, said method comprising the step of:
A. administering to a human or animal a composition comprising an epitope of a prefibrillar aggregate which forms in a human or animal contributing to an amyloid fibril formation wherein the amyloid fibril is substantially free of the epitope.

68. The method according to claim 67 wherein step A comprises recovering the monoclonal antibody from the human or animal.

69. A method for diagnosing a disease or condition in a human or animal subject, said disease or condition being characterized by the formation of amyloid deposits in neural tissue, said method comprising the step of:
A. combining tissue or fluid from the human or animal subject and a composition comprising or consisting of a monoclonal antibody, said monoclonal antibody being one that binds to a conformational epitope of a prefibrillar aggregate that contributes to amyloid fibril formation.

70. A method according to claim 69 wherein the disease or condition is selected from the group consisting of Alzheimer's, early onset Alzheimer's associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

71. A method according to claim 69 wherein the disease or condition is Alzheimer's Disease.

72. A method according to claim 69 wherein the tissue or fluid is cerebrospinal fluid.

73. A method for diagnosing a disease or condition in a human or animal subject, said disease or condition being characterized by the formation of amyloid deposits in neural tissue, said method comprising the step of:
A. combining tissue or fluid from a human or animal subject and a composition comprising a monoclonal antibody which binds to an epitope of a prefibrillar aggregate which forms in a human or animal contributing to an amyloid fibril formation wherein the amyloid fibril is substantially free of the epitope.

74. A method according to claim 73 wherein the disease or condition is selected from the group consisting of Alzheimer's Disease, early onset Alzheimer's Disease associated with Down's syndrome, SAA amyloidosis, hereditary Icelandic syndrome, multiple myeloma, and spongiform encephalopathies, including mad cow disease, sheep scrapie, and mink spongiform encephalopathy, Parkinson's disease, Huntington's disease, amyotropic lateral sclerosis, Creutzfeld Jakob disease, Gerstmann-Straussler-Scheinker syndrome, kuru, fatal familial insomnia, chronic wasting syndrome, familial amyloid polyneuropathy, frontotemporal dementia, type II diabetes, systemic amyloidosis, serum amyloidosis, British familial dementia, Danish familial dementia, macular degeneration and cerebrovascular amyloidosis.

75. A method according to claim 73 wherein the disease or condition is Alzheimers Disease.

76. A method according to claim 73 wherein the tissue or fluid is cerebrospinal fluid.

77. A diagnostic kit useful for detecting a disease or condition characterized by amyloid deposits in the central nervous system of a human or animal subject, said kit comprising:
a composition that consists of or comprises a monoclonal antibody which binds to a conformational epitope of a prefibrillar aggregate which forms in the human or animal subject and contributes to amyloid fibril formation.

78. A kit according to claim 77 wherein the monoclonal antibody is specific for a conformation-dependent epitope that is preferentially displayed by oligomeric conformations of Aβ and other amyloids.

79. A diagnostic kit useful for detecting a disease or condition characterized by amyloid deposits in the central nervous system of a human or animal subject, said kit comprising:
an isolated composition comprising a monoclonal antibody which binds to an epitope of a prefibrillar aggregate which contributes to amyloid fibril formation.

80. A kit according to claim 79 wherein the monoclonal antibody is specific for a conformation-dependent epitope that is preferentially displayed by oligomeric conformations of Aβ and other amyloids.

81. A method for treating or preventing Alzheimer's Disease and/or another amyloid disease which causes brain inflammation in a human or animal subject, said method comprising the steps of:
A) administering to the subject a therapeutically effective amount of a monoclonal antibody composition according to claim 1; and
B) administering to the subject an antiinflamatory agent in an amount that is effective to deter brain inflammation.

82. A method according to claim 81 wherein Step B comprises administering gold or a gold-containing compound to the subject in an amount that is therapeutically effective to decrease neural inflammation.

83. A method according to claim 81 wherein a colloidal gold preparation is administered in Step B.

84. A method according to claim 81 wherein the anti-inflammatory agent is combined with the monoclonal antibody.

85. A method according to claim 81 wherein the anti-inflammatory agent is separate from the monoclonal antibody.
